(19) United States
(12) Patent Application Publication
(10) Pub. No.: US 2024/0189667 A1
(43) Pub. Date: Jun. 13, 2024

(54) CABLE HEAD ADJUSTMENT APPARATUS AND METHOD

(71) Applicant: John M. Bird, Sparks, NV (US)

(72) Inventor: John M. Bird, Sparks, NV (US)

(21) Appl. No.: 18/500,882

(22) Filed: Nov. 2, 2023

Related U.S. Application Data
(63) Continuation-in-part of application No. 17/385,876, filed on Jul. 26, 2021, now Pat. No. 11,833,394.
Application No. 17/385,876 is a continuation of application No. 16/231,566, filed on Dec. 23, 2018, now Pat. No. 11,071,890.
Application No. 16/231,566 is a continuation of application No. 15/660,835, filed on Jul. 26, 2017, now Pat. No. 10,159,870.
Application No. 15/660,835 is a continuation of application No. 14/613,259, filed on Feb. 3, 2015, now Pat. No. 9,717,952.
Application No. 14/613,259 is a continuation of application No. 13/956,337, filed on Jul. 31, 2013, now Pat. No. 8,968,155.
(60) Provisional application No. 63/381,961, filed on Nov. 2, 2022.
(60) Provisional application No. 63/381,967, filed on Nov. 2, 2022.
(60) Provisional application No. 63/381,968, filed on Nov. 2, 2022.
(60) Provisional application No. 61/677,640, filed on Jul. 31, 2012.
(60) Provisional application No. 61/778,101, filed on Mar. 12, 2013.

(51) Int. Cl. A63B 24/00; A63B 21/00; A63B 21/005; A63B 23/035; A63B 23/12; A63B 71/00; G16H 20/30; G16Z 99/00

(52) U.S. Cl. CPC A63B 24/0087; A63B 21/005; A63B 21/0058; A63B 21/151; A63B 21/153; A63B 21/156; A63B 21/4043; A63B 23/03525; G16H 20/30; G16Z 99/00; A63B 21/0053; A63B 23/1263; A63B 23/1281; A63B 2024/0093; A63B 2071/0072; A63B 2220/13; A63B 2220/17; A63B 2220/30; A63B 2225/20; A63B 2225/50

(57) ABSTRACT

A movable cable head adjustment compensation assembly may include one or more of a first pulley configured to receive a head adjustment cable, a second pulley configured to receive a resistance cable, and a stabilizer. A movable cable head adjustment compensation assembly may include a connecting plate coupled to the first pulley, the second pulley, and the stabilizer. A cable head adjustment apparatus may include a cable head, a movable cable head adjustment compensation assembly, a head adjustment cable, a resistance cable, a cable drum, and a lower pulley.

### CROSS-REFERENCE TO RELATED APPLICATIONS

This application claims priority to U.S. Provisional Patent Application Nos. 63/381,961, filed on Nov. 2, 2022 entitled “ASSISTED RESISTANCE APPARATUS, SYSTEM, AND METHOD”, 63/381,967, filed on Nov. 2, 2022 entitled “PULSED MOTOR CONTROL SYSTEM AND METHOD”, and 63/381,968 filed on Nov. 2, 2022 entitled “MOTOR VOLTAGE MONITORING AND MOTOR TORQUE ADJUSTMENT SYSTEM AND METHOD”, and is further a continuation in part of U.S. application Ser. No. 17/385,876, filed Jul. 26, 2021, entitled “EXERCISE APPARATUS WITH MOTOR INDUCED RESISTANCE”, which is a continuation of U.S. application Ser. No. 16/231,566, filed Dec. 23, 2018, entitled “RESISTANCE APPARATUS, SYSTEM, AND METHOD”, which is a continuation of U.S. application Ser. No. 15/660,835, filed Jul. 26, 2017, entitled “RESISTANCE APPARATUS, SYSTEM, AND METHOD” (now U.S. Pat. No. 10,159,870), which is a continuation of U.S. application Ser. No. 14/613,259, filed Feb. 3, 2015, entitled “RESISTANCE APPARATUS, SYSTEM, AND METHOD” (now U.S. Pat. No. 9,717,952), which is a continuation of U.S. application Ser. No. 13/956,337, filed Jul. 31, 2013, entitled “RESISTANCE APPARATUS, SYSTEM, AND METHOD” (now U.S. Pat. No. 8,968,155), which claims priority to U.S. Provisional Patent Application No. 61/677,640, entitled “PROGRAMMABLE ELECTRONIC RESISTANCE SYSTEM AND METHOD OF USE,” filed on Jul. 31, 2012, and to U.S. Provisional Patent Application No. 61/778,101, entitled “PROGRAMMABLE ELECTRONIC RESISTANCE SYSTEM AND METHOD OF USE,” filed on Mar. 12, 2013, all of which are expressly incorporated by reference herein. In the event of any inconsistency between the priority applications recited above and this application, this application shall prevail.

### COPYRIGHT

A portion of the disclosure of this patent document contains or may contain material subject to copyright protection. The copyright owner has no objection to the photocopy reproduction of the patent document or the patent disclosure in exactly the form it appears in the Patent and Trademark Office patent file or records, but otherwise reserves all copyright rights.

### TECHNICAL FIELD

The present disclosure relates to resistance training systems and methods in general and, in one embodiment, to a system and method that provides for simulating physical weights (mass subject to gravity) through electrical and mechanical components with low torque level control of user experienced resistance through insertion of electronic resistance.

### BACKGROUND ART

There is a long-standing need for a mechanism for resistance machines that allows cable head adjustment while maintaining a 1:1 weight-stack-to-User resistance ratio. Typically, the ratio has been 2:1 where the weight stack weight selection was twice what the user desired (i.e. the user experienced a maximum of one-half the maximum weight stack weight). There is a need for a mechanism such that cable head adjustment does not include, for example, one or more of, a 2x maximum excursion cable length, 2x cable drum width, resistance motor activation, in order to adjust the cable head position.

Referring now to FIG. 1, the loop cable (sometimes called the resistance cable) was part of the interface between the user and the weight stack. The loop cable was pulled by the user during exercise. The loop cable typically made a complete loop beginning and ending at the cable head.

Historically, cable machines having adjustable cable heads, such as, for example, what is seen in many crossover machines, generally had a loop cable running from a connection at the rear of the cable head around a weight stack pulley and pulley array, exiting at the cable head where it interfaced to the User. As the cable head was moved up and down the adjustment post, the loop cable moved over the pulley array allowing cable head movement while the weight stack remained at rest. The cable head was then adjusted for position while the weight stack remained at rest.

Unfortunately, the loop cable and weight stack pulley mechanism resulted in a 1:2 reduction in weight to the user, such as, for example, if the user wanted 25 lbs of resistance, the weight stack had to be set for 50 lbs of weight. This was an inefficient implementation of weight stack material in terms of cost, shipping, gross weight, etc. Further, up to two times the cable length (i.e. maximum exercise cable excursion) was often needed to adjust the cable head from minimum position to maximum position (e.g., low to high and high to low) for either, or example, a chain drive or cable drum mechanism. If chain driven, the tower also typically needed to be two times the height. If cable drum driven, the drum typically needed to be two times the cable take up width. The motor often had to be activated for the cable head adjustment such that the cable could be spooled out, taken up, or both.

### SUMMARY OF THE INVENTION

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of this disclosure.

In some aspects, the techniques described herein relate to a movable cable head adjustment compensation assembly, including: a first pulley configured to receive a head adjustment cable; a second pulley configured to receive a resistance cable; a stabilizer; and a connecting plate coupled to the first pulley, the second pulley, and the stabilizer.

In some aspects, the techniques described herein relate to a cable head adjustment apparatus, including: a cable head; a movable cable head adjustment compensation assembly; a head adjustment cable; a resistance cable; a cable drum; and a lower pulley.

In some aspects, the techniques described herein relate to a cable head adjustment apparatus, further including a cable guide.

In some aspects, the techniques described herein relate to a cable head adjustment apparatus, further including a head adjustment cable adjuster.

It is to be understood that the foregoing is only a brief summary of some aspects of this specification. The present specification discloses many other novel features, problem solutions, and advantages. They will become apparent as this specification proceeds. Thus, the scope of a given claim is to be determined by the claim as issued and not by whether it addresses an issue set forth in the above Background or includes a feature set forth in this Summary.

### BRIEF DESCRIPTION OF THE DRAWINGS

A further understanding of the nature and advantages of the embodiments may be realized by reference to the following drawings. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 is a front view of a traditional cable head/weight stack;

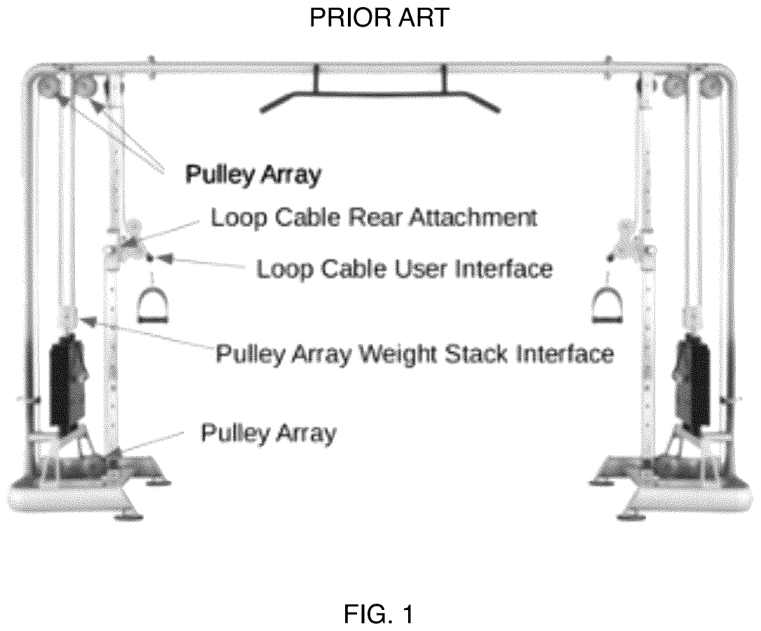

FIG. 2 is side view of a force cable single pulley arrangement;

FIG. 3 is a side view of a force cable single pulley arrangement example;

FIG. 4 is a side view of a cable head adustment compensation assembly;

FIG. 5 is an upper portion side view of a compensating assembly with cable routing;

FIG. 6 is a bottom portion side view of a compensating assembly with cable routing;

FIG. 7A is a side view of a compensation assembly and stabilizer;

FIG. 7B is a perspective view of the compensation assembly and stabilizer of FIG. 7A;

FIG. 8 is a perspective view of a top cable guide an bolted implementation;

FIG. 9A is a side view of a head adjustment cable adjuster turnbuckle bolt implementation;

and

FIG. 9B is a perspective view of the head adjustment cable adjuster turnbuckle bolt implementation of FIG. 9A.

### DETAILED DESCRIPTION

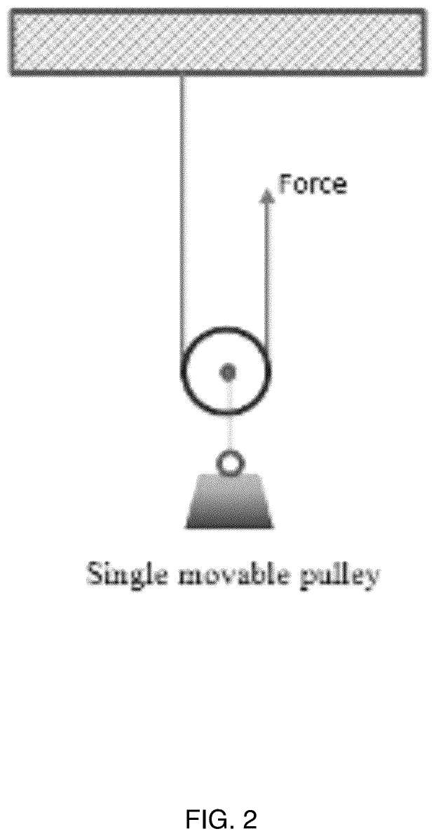

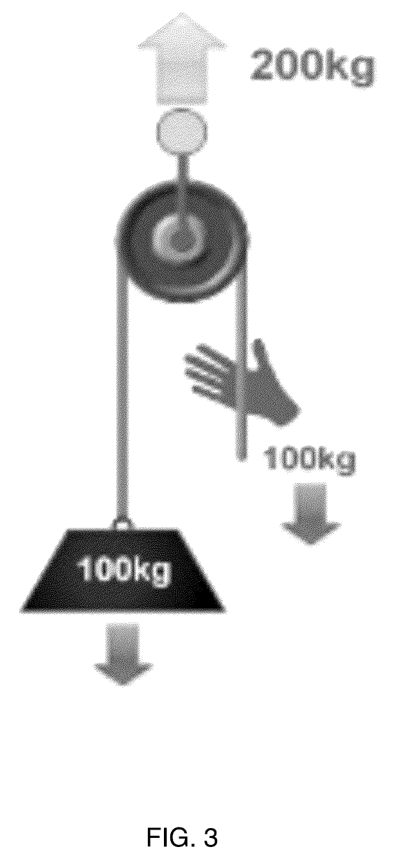

The one or more of the aforementioned issues are addressed by a better use of pulleys and pulley/cable properties. Referring now to FIG. 2 and FIG. 3, a single pulley can be used to translate cable travel (distance) and cable force. The cable head needs to move without the loop or resistance cable binding or slacking. The single movable pulley force cable is fixed at one end and that the weight will move half the distance of the force cable.

The desired cable head adjustment is achieved by substituting a second pulley for the weight and adding cabling to that second pulley. There are now 2 cables that interact via the cable head adustment compensation assembly, which will move up and down as the cable head is adjusted down and up, noting the inverted motion. The interaction between the force cable, which this specification will refer to as the head adjustment cable, and the secondary cable, which this specification will refer to as the resistance cable, compensates for cable head adjustment, addressing the aforementioned problems.

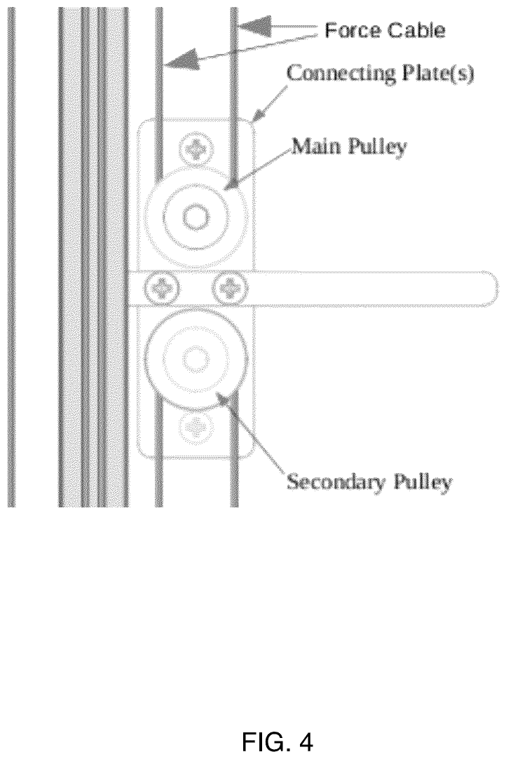

Referring now to FIG. 4, in some embodiments, retrofitting an existing loop cable design can involve breaking the traditional loop cable connection such that the loop cable output is no longer directly connected to the cable head. This is now the force cable (head adjustment cable) and can run from the cable head connection over (or under) a pulley, referred to here as the main pulley, to an anchor point attached to the machine. This anchor point can be fixed or adjustable. The main pulley can be connected to a secondary pulley via one or more connecting plate.

The cable head adjustment compensation assembly includes a main pulley, a secondary pulley, a stabilizer, and one or more connecting plate. In some instances, the stabilizer can assist, at least in part, in one or more of reducing or preventing the cable head adjustment compensation assembly from moving or making noise when the exercise apparatus is in use or moved, and keeping the cable head adjustment compensation assembly centered during cable head adjustment, which can be of significant value if the cable head adjustment compensation assembly transitions between, for example, narrow support braces. In some implementations, the cable head adjustment compensation assembly moves half the distance of the head adjustment cable.

In some implementations, the weight stack interface pulley is removed and directly connected to the resistance cable. Although the resistance cable exits the cable head in a standard fashion, it can do so indirectly, passing over the secondary pulley, then over a lower fixed pulley, and exiting the cable head. The lengths of the head adjustment cable and the loop cable can be determined by first putting the cable head at either full top or full bottom position, then connecting the head adjustment cable to the cable head and running it over the main pulley to the machine connection point. Next, connecting the loop cable to the weight stack and running it over the secondary pulley, the lower pulley, and out the cable head, such as to the user, adjusting for any cable connection specifics, such as, for example, cable thimbles.

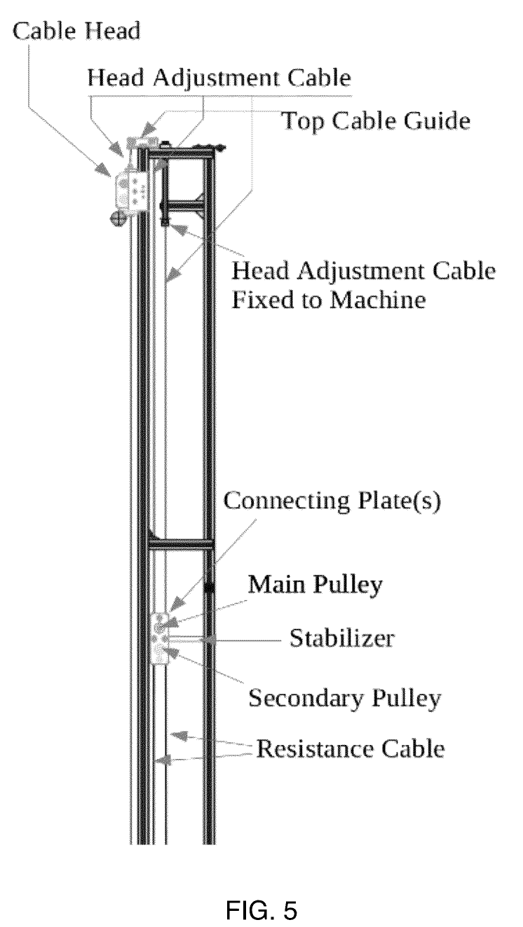

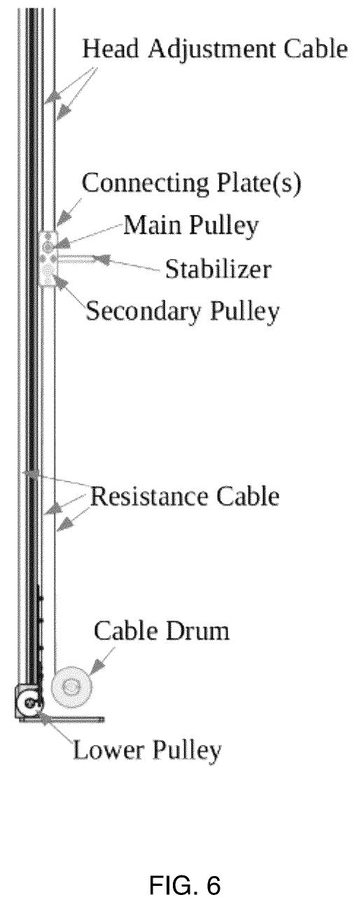

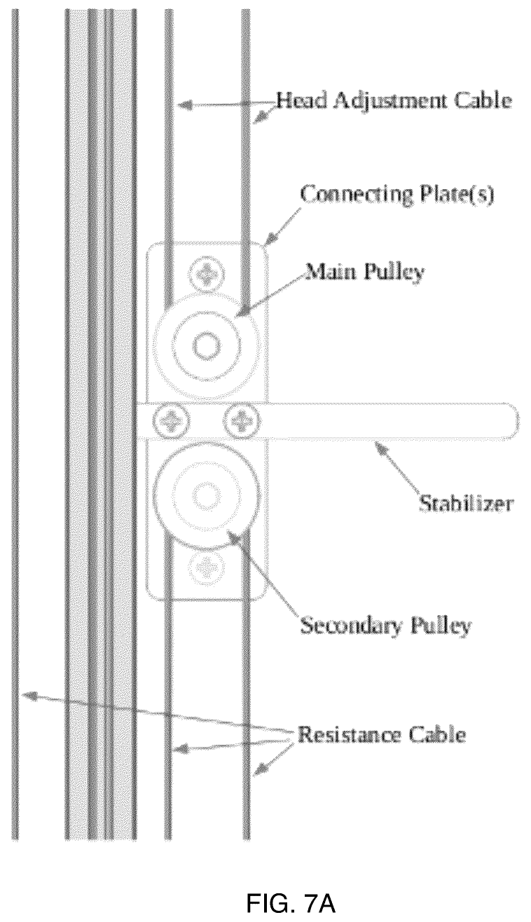

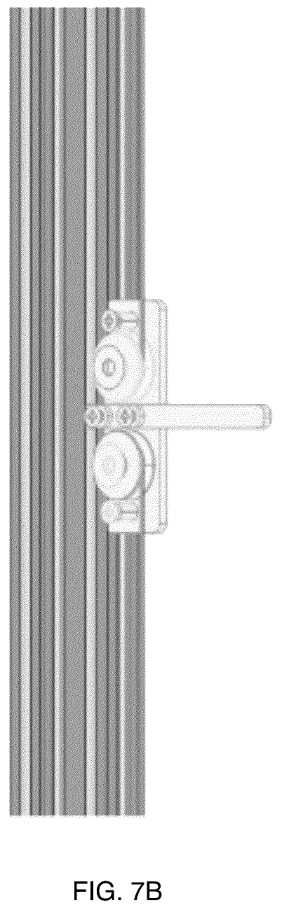

Referring now to FIG. 5, FIG. 6, FIG. 7A, and FIG. 7B, in some instances, there are two cables, namely, the head adjustment cable and the resistance cable, which implementation can use a cable drum. The head adjustment cable and resistance cable move the cable head adustment compensation assembly as they are pulled via the cable head. As the cable head is moved downward, it pulls on the head adjustment cable which pulls the cable head adustment compensation assembly upward, which in turn pulls (i.e., retracts) the resistance cable. Conversely, as the cable head is moved upward, it pulls on the resistance cable which pulls the cable head adustment compensation assembly downward, which in turn pulls (i.e., retracts) the head adjustment cable. These cable motion offsets allow the cable head to be adjusted as desired while addressing the problems identified previously.

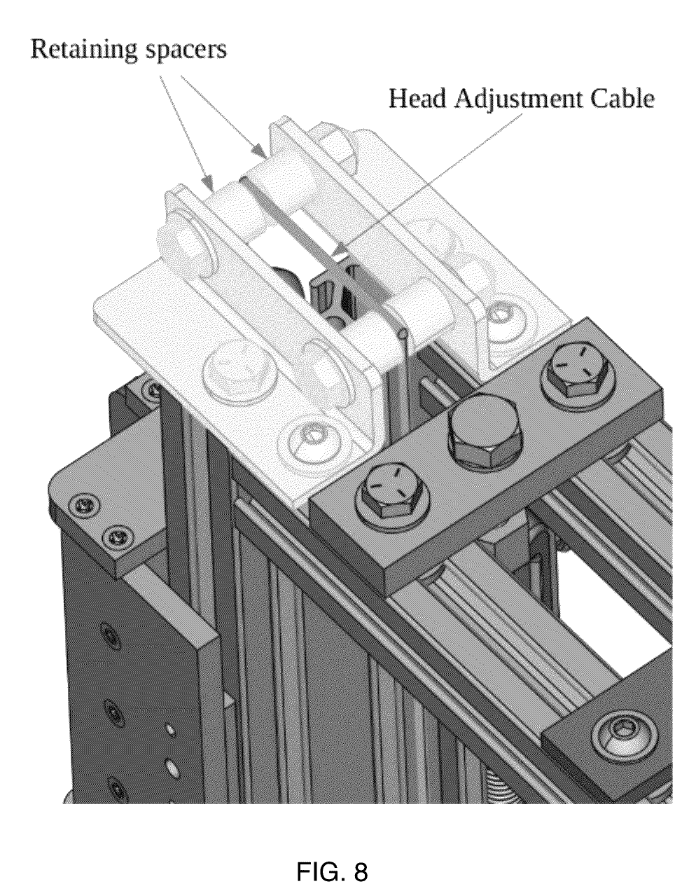

Referring now to FIG. 8, in some embodiments, pulleys are absent for the head adjustment cable as that cable might only move during cable head adjustment, and not during exercise. The cable head can then be adjusted during non-exercise time. For motor driven resistance, the machine can be off or in a non-exercise or idle mode where the resistance cable is lightly loaded.

In some implementations, a top cable guide uses bolts instead of pulleys. The head adjustment cable rides on the smooth part of the connecting bolts and does not use a pulley for operation. Retaining spacers can keep the cable from wandering from side to side.

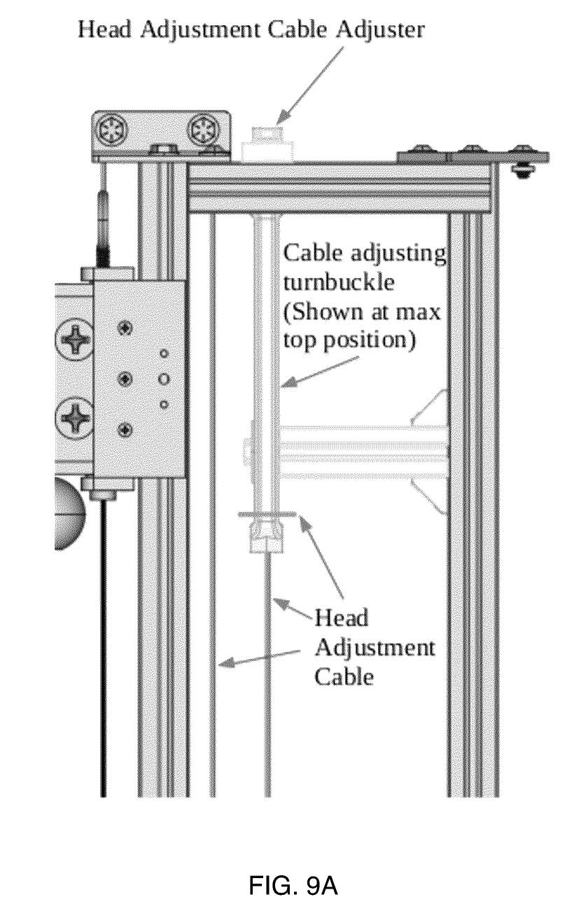

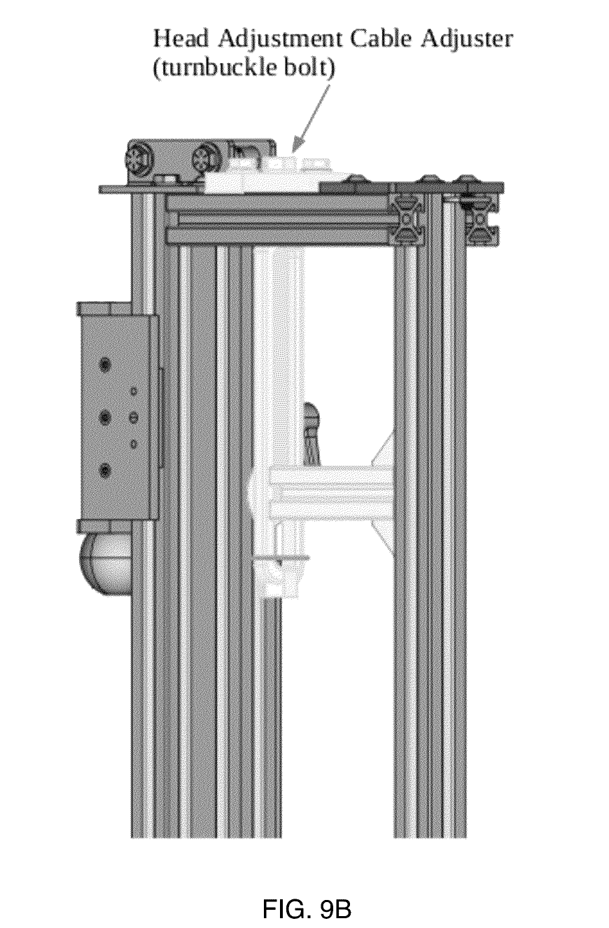

Referring now to FIG. 9A and FIG. 9B, with head adjustment cable adjuster, the head adjustment cable can be adjusted for length, resulting in the resistance cable being pulled tighter or loosened with respect to the cable head.

Clause 1. A movable cable head adjustment compensation assembly, comprising: a first pulley configured to receive a head adjustment cable; a second pulley configured to receive a resistance cable; a stabilizer; and a connecting plate coupled to the first pulley, the second pulley, and the stabilizer.

Clause 2. A cable head adjustment apparatus, comprising: a cable head; a movable cable head adjustment compensation assembly; a head adjustment cable; a resistance cable; a cable drum; and a lower pulley.

Clause 3. The cable head adjustment apparatus of claim 2, further comprising a cable guide.

Clause 4. The cable head adjustment apparatus of claim 2, further comprising a head adjustment cable adjuster.

Although the described embodiments of the present invention are believed to represent the best mode of the present invention, it should be understood that many described components of the present invention have known functional equivalents. Additionally, orientations, proportions and shapes used within the description may be modified by a person skilled in the art. All such modifications and deviations are intended to be covered by the scope of the invention.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” “approximately,” “nearly,” or the like, it will be understood that the particular value forms another aspect. It will be further understood that the endpoints of each of the ranges are significant both in relation to the other endpoint, and independently of the other endpoint.

Conditional language, such as “can,” “could,” “might,” or “may,” unless specifically stated otherwise, or otherwise understood within the context as used, is generally intended to convey that certain implementations include or do not include, certain features, elements, and/or steps. Thus, such conditional language is not generally intended to imply that features, elements, and/or steps are in any way required for one or more implementations.

Conjunctive language, such as the phrase “at least one of X, Y, and Z.” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Several illustrative implementations of the cable head adjustment apparatus and method have been disclosed. Although this disclosure has been described in terms of certain illustrative implementations and uses, other implementations and other uses, including implementations and uses which do not provide all of the features and advantages set forth herein, are also within the scope of this disclosure. Components, elements, features, acts, or steps can be arranged or performed differently than described and components, elements, features, acts, or steps can be combined, merged, added, or left out in various implementations. All possible combinations and subcombinations of elements and components described herein are intended to be included in this disclosure. No single feature or group of features is necessary or indispensable.

Certain features that are described in this disclosure in the context of separate embodiments can also be implemented in combination in a single embodiment. Conversely, various features that are described in the context of a single implementation also can be implemented in multiple embodiments separately or in any suitable subcombination. Moreover, although features may be described above as acting in certain combinations, one or more features from a claimed combination can in some cases be excised from the combination, and the combination may be claimed as a subcombination or variation of a subcombination.

Any portion of any of the steps, processes, structures, and/or devices disclosed or illustrated in one implementation or example in this disclosure can be combined or used with (or instead of) any other portion of any of the steps, processes, structures, and/or devices disclosed or illustrated in a different implementation, flowchart, or example. The implementations and examples described herein are not intended to be discrete and separate from each other. Combinations, variations, and some implementations of the disclosed features are within the scope of this disclosure.

While operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Additionally, the operations may be rearranged or reordered in some implementations. Also, the separation of various components in the implementations described above should not be understood as requiring such separation in all implementations, and it should be understood that the described components and systems can generally be integrated together in a single product or packaged into multiple products. Additionally, some implementations are within the scope of this disclosure.

Further, while illustrative embodiments have been described, any implementations having equivalent elements, modifications, omissions, and/or combinations are also within the scope of this disclosure. Moreover, although certain aspects, advantages, and novel features are described herein, not necessarily all such advantages may be achieved in accordance with any particular implementation. For example, some implementations within the scope of this disclosure achieve one advantage, or a group of advantages, as taught herein without necessarily achieving other advantages taught or suggested herein. Further, some implementations may achieve different advantages than those taught or suggested herein.

Additionally, any methods described herein may be practiced using any device suitable for performing the recited steps.

In summary, various implementations and examples of cable head adjustment apparatus and method have been disclosed. This disclosure extends beyond the specifically disclosed implementations and examples to other alternative implementations and/or other uses of the implementations, as well as to certain modifications and equivalents thereof. Moreover, this disclosure expressly contemplates that various features and aspects of the disclosed implementations can be combined with, or substituted for, one another. Accordingly, the scope of this disclosure should not be limited by the disclosed implementations described above, but should be determined only by a fair reading of the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present systems and methods and their practical applications, to thereby enable others skilled in the art to best utilize the present systems and methods and various embodiments with various modifications as may be suited to the particular use contemplated.

Unless otherwise noted, the terms “a” or “an,” as used in the specification are to be construed as meaning “at least one of.” In addition, for ease of use, the words “including” and “having,” as used in the specification, are interchangeable with and have the same meaning as the word “comprising.” In addition, the term “based on” as used in the specification is to be construed as meaning “based at least upon.”

1. A movable cable head adjustment compensation assembly, comprising:
a first pulley configured to receive a head adjustment cable;
a second pulley configured to receive a resistance cable;
a stabilizer; and
a connecting plate coupled to the first pulley, the second pulley, and the stabilizer.

2. A cable head adjustment apparatus, comprising:
a cable head;
a movable cable head adjustment compensation assembly;
a head adjustment cable;
a resistance cable;
a cable drum; and
a lower pulley.

3. The cable head adjustment apparatus of claim 2, further comprising a cable guide.

4. The cable head adjustment apparatus of claim 2, further comprising a head adjustment cable adjuster.
